(19) United States
(12) Patent Application Publication
(10) Pub. No.: US 2012/0071844 A1
(43) Pub. Date: Mar. 22, 2012

(54) SUCTION BUTTON ASSEMBLY FOR ENDOSCOPE

(76) Inventor: Kenji YAMANE, Kanagawa (JP)

(21) Appl. No.: 13/238,741

(22) Filed: Sep. 21, 2011

(30) Foreign Application Priority Data
Sep. 22, 2010 (JP) 2010-211859

(51) Int. Cl. A61M 1/00

(52) U.S. Cl. 604/319

(57) ABSTRACT

An endoscope includes an operation unit, an elongated tube, a distal opening, a suction channel, and a discharge conduit. A suction button assembly has a valve structure for changing over between the discharge conduit and the suction channel for communication and shut-off. The suction button assembly includes a plunger, a first stem end, and a valve opening of the plunger. A cap device is coupled to the first stem end and to the first cylinder end of a cylinder housing in the operation unit, and around the first stem end. A first regulating device prevents the plunger from rotating in the cap device. A second regulating device prevents the cap device from rotating about an axis of the cylinder housing, to keep the valve opening aligned with a flow opening of a cylinder passage of the cylinder housing.

### BACKGROUND OF THE INVENTION

1. Field of the Invention

The present invention relates to a suction button assembly for an endoscope. More particularly, the present invention relates to a suction button assembly for an endoscope, in which suction of fluid from a body cavity can be controlled precisely.

2. Description Related to the Prior Art

An endoscope includes an operation unit and an elongated tube extending distally from the operation unit. The elongated tube includes a suction channel and a distal opening. The suction channel extends in an axial direction. The distal opening is formed in a distal end face of the elongated tube, as a distal end of the suction channel. A suction button assembly is incorporated in the operation unit. A specific example of the suction channel is an instrument channel, which is used for penetration of a forceps or other medical instrument and for supply of washing water or the like. A portion of the suction channel is a branch of the instrument channel, and is connected to the suction button assembly.

In addition to the suction channel, a discharge conduit for suction toward a negative pressure source is connected to the suction button assembly. An example of the negative pressure source is a suction pump. The suction button assembly, when depressed manually by a doctor or operator, connects the discharge conduit to the suction channel for carry out suction through the distal opening. The suction button assembly, when left without the depression, disconnects the discharge conduit from the suction channel to discontinue the suction. See JP-A 2007-252589.

In FIGS. 14A and 14B, a known example of suction button assembly 108 is illustrated, and includes a cylinder housing 110, a plunger 115 and a cap 116. The cylinder housing 110 is contained in the operation unit of the endoscope. A distal opening of the cylinder housing 110 is open in the outside of the operation unit. A proximal opening of the cylinder housing 110 is connected to a suction channel 109. The cylinder housing 110 has a cylinder passage 111 (cylinder bore) and a cylinder opening 113. The plunger 115 is contained in the cylinder passage 111 in a slidable manner, and has a stem end 114 protruding from the cylinder opening 113. The cap 116 connects the end of the plunger 115 to the end of the cylinder housing 110, and biases the stem end 114 in an upward direction.

An opening 118 for a negative pressure source is formed to open intermediately in the cylinder passage 111. A discharge conduit 117 for a negative pressure source is disposed for becoming connected with the opening 118. The plunger 115 includes a valve opening 119, an input valve port 120, and a flow channel 121. The input valve port 120 is open in its lower wall. The flow channel 121 extends between the valve opening 119 and the input valve port 120.

In FIG. 14A, an initial state of the suction button assembly 108 is illustrated. The stem end 114 is free before being depressed. A position of the valve opening 119 of the plunger 115 is higher than the opening 118 so that an outer surface of the plunger 115 closes the opening 118. The discharge conduit 117 is disconnected from the suction channel 109.

In FIG. 14B, a depressed state of the suction button assembly 108 is illustrated. The stem end 114 is pushed at a predetermined depth into the cylinder opening 113. The valve opening 119 is positioned in front of the opening 118, for the suction channel 109 to communicate with the discharge conduit 117 through the flow channel 121. Fluid drawn through the suction channel 109 into the cylinder passage 111 is passed through the flow channel 121 and the valve opening 119 and reaches the opening 118, to flow through the discharge conduit 117 to the outside of the endoscope.

If the valve opening 119 is not opposed to the opening 118 according to rotation of the plunger 115 about its axis, a passage for flow of fluid between the valve opening 119 and the opening 118 becomes very narrow. This may cause failure in passage of relatively large solid particles in the fluid through the passage. JP-A 2009-045126 and JP-A 2007-190054 therefore disclose the suction button assembly including a plunger and a regulating projection disposed to project from the plunger for preventing rotation. A flow channel has an inner surface, and an engaging groove formed to retract from the inner surface, disposed to extend in a longitudinal direction of the plunger, for receiving engagement of the regulating projection in a slidable manner. Thus, it is possible to regulate rotation between the cylinder and the plunger to oppose a lateral opening to an opening for the negative pressure source. Force for suction of the suction button assembly can be maximized. In short, solid particles passable in the valve assembly can have a larger size.

In the suction button assembly of JP-A 2009-045126 and JP-A 2007-190054, however, the regulating projection of the plunger is engaged with the engaging groove of the inner surface of the flow channel. Resistance of friction occurs in contact of the regulating projection with the inner surface of the engaging groove when the plunger moves in the flow channel. The resistance of the friction may be so large that failure of the plunger will occur seriously, for example the plunger will fail to return to its original position even though the cap device biases upon leaving the plunger without the depression.

### SUMMARY OF THE INVENTION

In view of the foregoing problems, an object of the present invention is to provide a suction button assembly for an endoscope, in which suction of fluid from a body cavity can be controlled precisely.

In order to achieve the above and other objects and advantages of this invention, a suction button assembly for an endoscope is provided, the endoscope including an operation unit, a section of an elongated tube for entry in a body cavity, and a suction channel and a discharge conduit disposed with the operation unit, wherein a distal opening is formed in a distal end of the elongated tube, the suction channel is disposed to extend to the distal opening, the discharge conduit is connected to a negative pressure source. In the suction button assembly, a cylinder housing is disposed on the operation unit, and includes a cylinder passage, a valve engaging surface and a flow opening, the cylinder passage being formed through the cylinder housing, the valve engaging surface being disposed in the cylinder passage, the flow opening formed in a wall of the cylinder passage, the cylinder passage having first and second cylinder openings, the first cylinder opening being connected to the suction channel, the valve engaging surface being tapered with an inner diameter decreasing from the first cylinder opening toward the second cylinder opening, the discharge conduit extending from the flow opening. A plunger is contained in the cylinder passage in a slidable manner, and includes a stem end, a valve head and a flow channel, the stem end protruding from the second cylinder opening, the valve head being movable to and from the valve engaging surface for contact, the flow channel being opposed to the flow opening, wherein the plunger is in a closed position when the stem end is free from being depressed, and is in an open position when the stem end is depressed, and the valve head, when the plunger is in the closed position, contacts the valve engaging surface for interruption between the suction channel and the discharge conduit, and when the plunger is in the open position, leaves from the valve engaging surface for communication between the suction channel and the discharge conduit in cooperation with the flow channel aligned with the flow opening. A cap device is secured to the stem end and the cylinder housing, for covering the stem end. A first regulating device prevents the plunger from rotating in the cap device. A second regulating device prevents the cap device from rotating about an axis of the cylinder housing, whereby keeping the flow channel aligned with the flow opening.

Preferably, the first regulating device includes a first regulating portion formed with the plunger. A second regulating portion is formed with the cap device, for engagement with the first regulating portion.

Preferably, the first regulating portion is a recess portion formed in the plunger, and the second regulating portion is a projection disposed to project from an inner surface of the cap device toward the recess portion.

Preferably, the second regulating device includes a third regulating portion formed with the cylinder housing. A fourth regulating portion is formed with the cap device, for engagement with the third regulating portion.

Preferably, the third regulating portion is a recess portion formed in the cylinder housing, and the fourth regulating portion is a projection disposed to project from an inner surface of the cap device toward the recess portion.

Preferably, the flow opening is formed in the valve engaging surface, and when the plunger is in the closed position, is closed by the valve head, and when the plunger is in the open position, is open upon separation of the valve head.

Preferably, the flow channel is formed through the plunger, and includes a flow port, formed at an end of the plunger, and open in the cylinder passage. A valve opening is formed in a wall of the plunger, and opposed to the flow opening when the plunger is in the open position.

Preferably, the valve head is disposed at an end of the plunger opposite to the stem end. The flow channel has an inner diameter increasing toward the flow port in the valve head.

Preferably, the cylinder passage further includes a first cylinder passage, disposed between the valve engaging surface and the first cylinder opening, and connected to the suction channel. A second cylinder passage is disposed between the valve engaging surface and the second cylinder opening, has a smaller inner diameter than the first cylinder passage, for containing the stem end in a slidable manner.

Preferably, a maximum outer diameter of the valve head is smaller than an inner diameter of the first cylinder passage.

In one preferred embodiment, the flow opening is disposed between the valve engaging surface and the second cylinder opening. The flow channel is formed between the valve head and the stem end, and kept opposed to the flow opening.

Preferably, the valve head is disposed at an end of the plunger opposed to the stem end. The flow channel is a longitudinal cutout formed linearly to extend in a plunger longitudinal direction. The cylinder passage is cylindrical with one inner diameter.

Preferably, furthermore, a vent channel is formed in the cylinder housing, disposed to extend to the discharge conduit, opened for venting of the discharge conduit when the plunger is in the closed position, and closed by the cap device when the plunger is in the open position.

Preferably, a vent opening is formed in the cap device, and a gap is formed between the second cylinder opening and the plunger when the plunger is in the open position, for venting through the vent opening in the cap device, and the gap is closed by the plunger when the plunger is in the closed position.

Preferably, the cap device is formed from resilient material and deformable resiliently upon slide of the plunger.

Preferably, the cap device includes a cap top portion of a disk shape. A cap skirt is formed to project in a downward direction from an edge of the cap top portion, having a lower end secured to the cylinder housing, for compressing in the downward direction when depressing force is exerted on the plunger to the open position, and for extending with return force thereof when the plunger is released from the depressing force, to return the plunger to the closed position.

Preferably, furthermore, an upper cylinder surface is disposed on the cylinder housing, and opposed to the cap device. A lower wall is disposed under the cap device, received by the upper cylinder surface upon sliding of the plunger stem in the downward direction, for preventing sliding thereof beyond a predetermined shift.

Preferably, the negative pressure source is a suction pump.

In another preferred embodiment, a suction button assembly of a valve structure for an endoscope includes a cylinder housing. A cylinder passage is formed through the cylinder housing. A plunger is contained in the cylinder passage, and slidable between open and closed positions. A suction channel is formed with the cylinder housing to extend in an upstream direction from the cylinder passage. A discharge conduit is formed with the cylinder housing to extend in a downstream direction from the cylinder passage. A flow opening is formed in a wall of the cylinder passage, the discharge conduit extending from the flow opening. A flow channel is formed in the plunger, for communicating the suction channel with the discharge conduit through the flow opening when the plunger is in the open position. A cap device is secured to an upper end of the plunger, for covering the plunger over the cylinder housing. A first regulating device prevents the plunger from rotating in the cap device. A second regulating device prevents the cap device from rotating about an axis of the cylinder housing, to keep the flow channel aligned with the flow opening.

Preferably, furthermore, a second opening is formed at an end of the flow channel, and opposed to the first opening when the plunger is in the open position.

Preferably, the plunger includes a plunger stem, disposed to project from the cylinder passage, for sliding in upward and downward directions through the cylinder passage. A valve sleeve is formed to extend from a stem end of the plunger stem, has the flow channel, set in the open position when the plunger stem slides in the downward direction, and set in the closed position when the plunger stem slides in the upward direction.

In one preferred embodiment, furthermore, a valve engaging surface is formed with the cylinder passage at a predetermined width. A valve head is disposed on the plunger and under the flow channel, shaped in association with the valve engaging surface, for opening the valve engaging surface when the plunger is in the open position for communication between the suction channel and the flow channel, and for contacting and closing the valve engaging surface when the plunger is in the closed position for shut-off between the suction channel and the flow channel.

Preferably, the flow channel extends in a longitudinal direction of the plunger, and slides in registration with the first opening.

Consequently, suction of fluid from a body cavity can be controlled precisely, because the first and second regulating devices can cooperate to prevent the cap device from rotating on the plunger and on the cylinder housing.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a vertical section illustrating a suction button assembly of the endoscope;

FIG. 3 is a vertical section illustrating the suction button assembly at the time of depression;

FIG. 4A is a vertical section illustrating a cylinder housing;

FIG. 4B is a perspective view illustrating the cylinder housing;

FIG. 5A is a perspective view illustrating a plunger;

FIG. 5B is a rear perspective view illustrating the plunger;

FIG. 6 is a vertical section illustrating a sliding valve sleeve in enlargement;

FIG. 7 is a perspective view, partially broken illustrating a cap device;

FIG. 8A is a vertical section illustrating a closed state with the sliding valve sleeve;

FIG. 8B is a vertical section illustrating an open state of a seat opening;

FIG. 9A is a vertical section illustrating another preferred suction button assembly;

FIG. 9B is a vertical section illustrating a portion of the suction button assembly in enlargement;

FIG. 10 is a vertical section illustrating the suction button assembly;

FIG. 11 is a perspective view illustrating the cylinder housing;

FIG. 12A is a perspective view illustrating the plunger;

FIG. 12B is a rear perspective view illustrating the plunger;

FIG. 13A is a perspective view illustrating the cap device;

FIG. 13B is a rear perspective view illustrating the cap device;

FIG. 14A is a vertical section illustrating a suction button assembly of a known structure;

FIG. 14B is a vertical section illustrating the suction button assembly of the known structure at the time of depression.

### DETAILED DESCRIPTION OF THE PREFERRED

### Embodiment(s) of the Present Invention

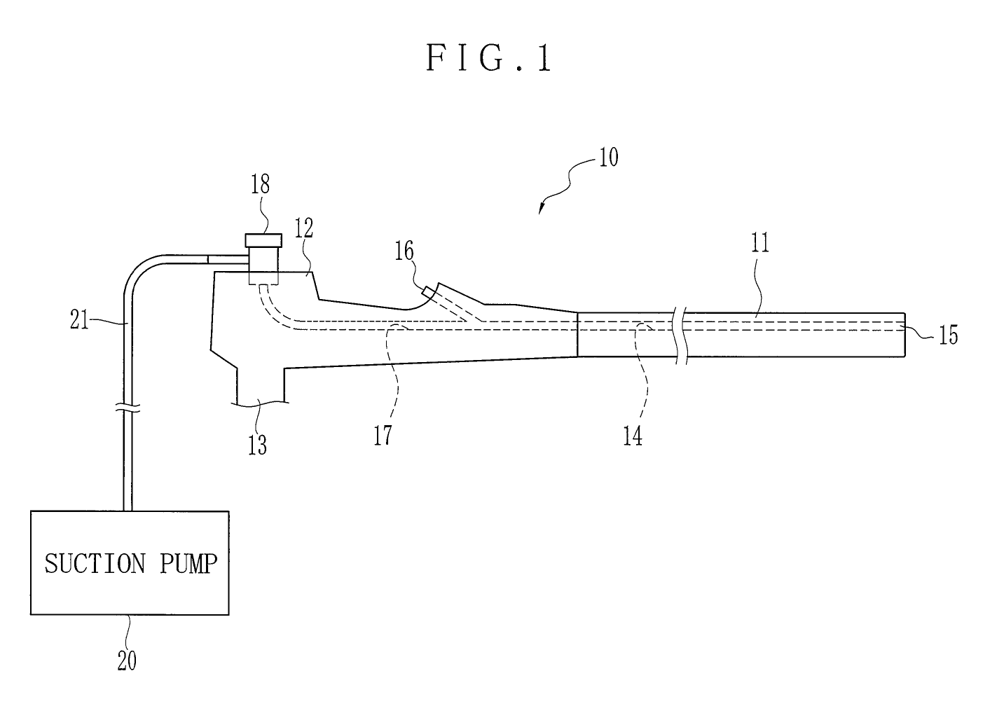

In FIG. 1, an endoscope 10 is a bronchoscope, and includes an elongated tube 11 or guide tube, an operation unit 12 and a universal cable 13. The elongated tube 11 is entered in a bronchus as a body cavity. The operation unit 12 is disposed at a proximal end of the elongated tube 11. An endoscope system for use includes a processing apparatus (not shown) and a light source apparatus (not shown). The universal cable 13 is connected to those apparatuses.

An instrument channel 14 is formed in the elongated tube 11 for entry of a forceps or other instrument for treatment. A distal instrument opening 15 is disposed at a distal end of the instrument channel 14, and open in a distal surface of the elongated tube 11. A proximal instrument opening 16 is disposed at a proximal end of the instrument channel 14, and open in the operation unit 12. A seal cap (not shown) is fitted in the proximal instrument opening 16 for closing at a normal time before entry of the instrument. Note that a syringe (not shown) can be connected with the proximal instrument opening 16 for injecting washing water such as physiological saline water. The water flows through the instrument channel 14 and is ejected by the distal instrument opening 15.

Windows (not shown) are formed in the distal surface of the elongated tube 11 in addition to the distal instrument opening 15, including an imaging window and lighting window. An image pickup device (not shown) is disposed behind the imaging window. An optical fiber cable (not shown) is disposed to extend from the lighting window. Signal lines and the optical fiber cable for the image pickup device are disposed to extend through the elongated tube 11 and the universal cable 13, and connected to the processing apparatus and light source apparatus.

The instrument channel 14 is used for transfer of fluid from the distal instrument opening 15 by suction, such as blood, waste fluid with mixed solid particles, or other body fluid. A suction channel 17 is formed in the operation unit 12 and extends as a branch of the instrument channel 14. A suction button assembly 18 or shut-off valve assembly of the invention is incorporated in the operation unit 12. The suction channel 17 extends to the suction button assembly 18.

A negative pressure source 20 or suction pump is installed externally. A discharge conduit 21 of the operation unit 12 is a flow line from the suction button assembly 18 to the negative pressure source 20. When the suction button assembly 18 is depressed or left without depression, a path is changed over for opening and closing between the suction channel 17 and the discharge conduit 21. The negative pressure source 20 is always used for suction during the imaging with the endoscope.

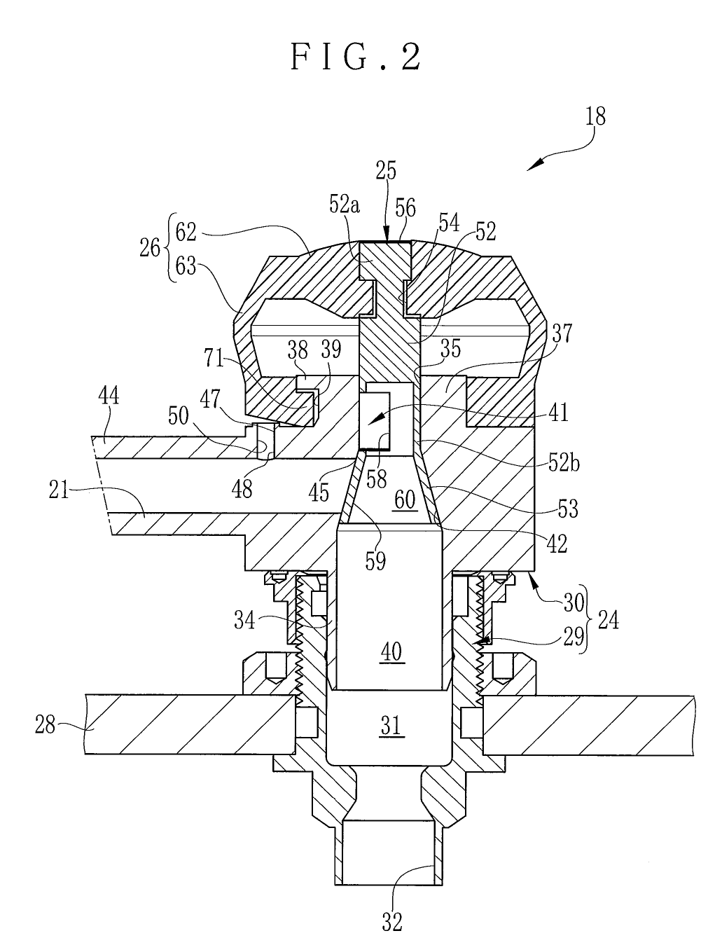

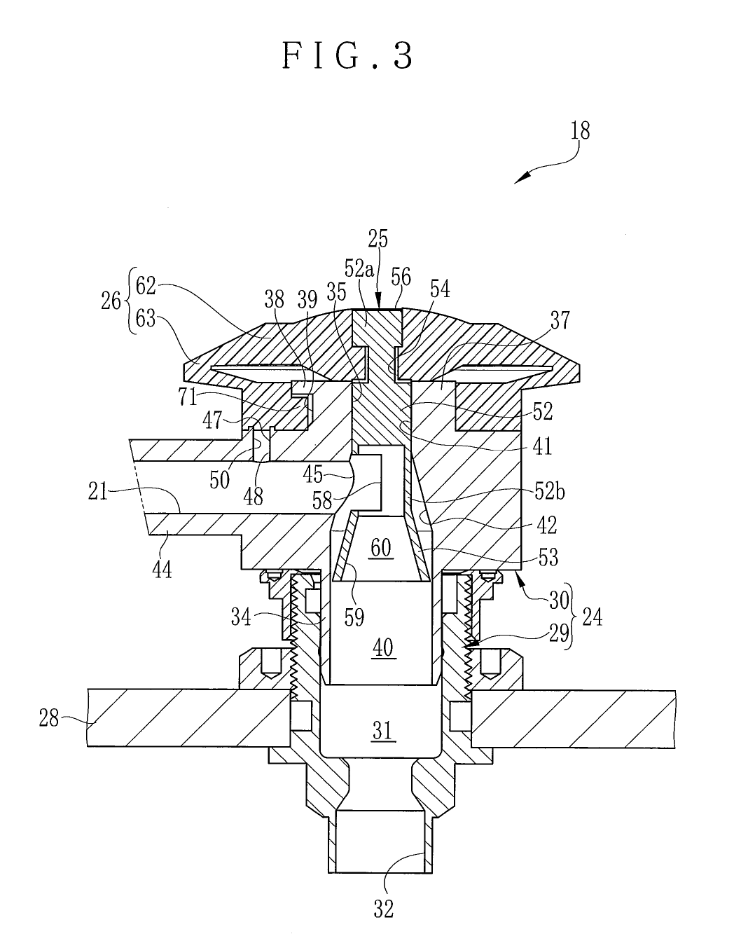

In FIG. 2, an initial state without depression is illustrated. In FIG. 3, a depressed state is illustrated. The suction button assembly 18 includes a housing device 24 or cylinder device, a plunger 25 and a cap device 26. The housing device 24 is attached to the operation unit 12. The plunger 25 is contained in the housing device 24. The cap device 26 connects the plunger 25 to the housing device 24 and covers those.

The operation unit 12 has a handle housing 28. The housing device 24 includes a support sleeve 29 or valve housing, and a cylinder housing 30 or valve guide. The support sleeve 29 is fixedly secured to the handle housing 28. The cylinder housing 30 is fixedly connected with the support sleeve 29. Note that an upper side in the drawings is referred to as an output side or upper side, a lower side being referred to as an input side.

The support sleeve 29 has an output end protruding to the outside of the handle housing 28, and an input end protruding to the inside of the handle housing 28. The support sleeve 29 is fixedly secured to the handle housing 28. A flow chamber 31 in the support sleeve 29 extends in the longitudinal direction. A valve port 32 for suction (lower cylinder end) is formed at an input end of the support sleeve 29 for connection with the suction channel 17. The flow chamber 31 communicates with the suction channel 17 by means of the valve port 32. An output end of the flow chamber 31 is connected with the cylinder housing 30.

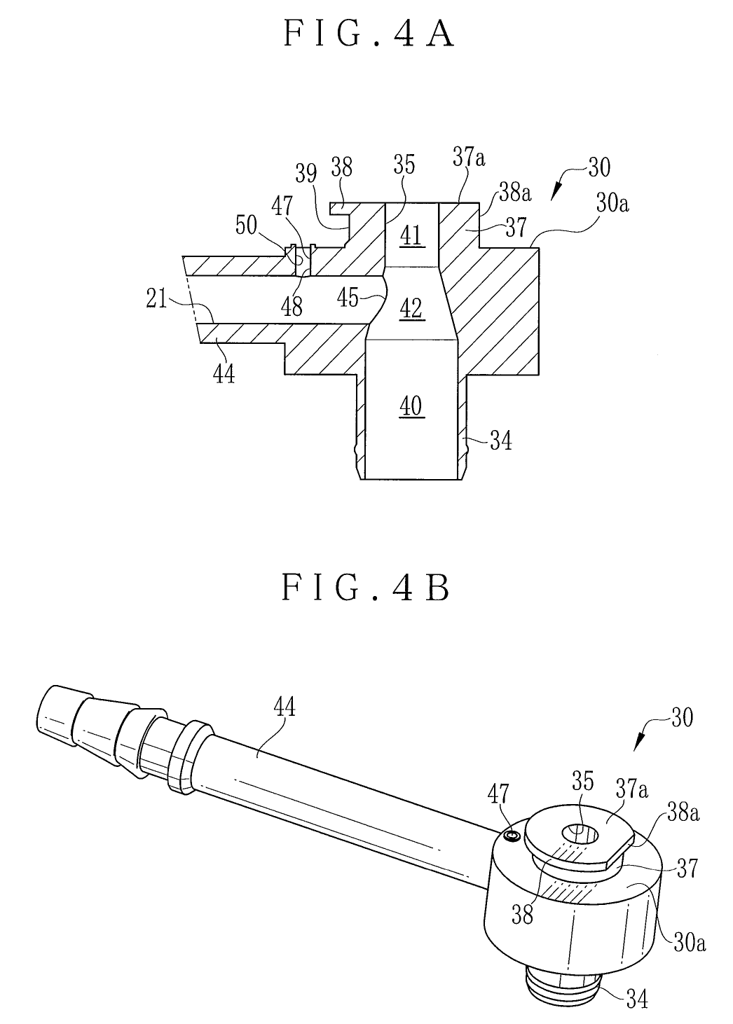

A flow sleeve 34 is a portion at input end of the cylinder housing 30. An output end of the support sleeve 29 is connected with the flow sleeve 34, which is concentric with the flow chamber 31. A cylinder opening 35 is formed in an upper cylinder end of the cylinder housing 30, and causes an upper end of the plunger 25 to protrude. See FIGS. 4A and 4B.

An end surface 30a is disposed at an end of the housing device 24 of FIGS. 4A and 4B. A cylinder sleeve 37 is disposed to project from the end surface 30a for connection with the cap device 26. In FIGS. 4A and 4B, an upper cylinder surface 37a is disposed at an end of the cylinder sleeve 37. The cylinder opening 35 is open in the upper cylinder surface 37a. A bonnet flange 38 projects from an outer surface of the cylinder sleeve 37. In FIG. 4B, a cap regulating surface 38a in a regulating device is formed in the bonnet flange 38 by chamfering flatly, and prevents the cap device 26 from rotating. In FIG. 4A, a sealing groove 39 is defined annularly by plural surfaces including a lower surface of the bonnet flange 38, the outer surface of the cylinder sleeve 37 and the end surface 30a, for connection with the cap device 26.

In FIG. 4A, the cylinder housing 30 includes a first cylinder passage 40, a second cylinder passage 41 (cylinder bores) and a conically tapered passage 42 or valve engaging surface. The first cylinder passage 40 extends in parallel with the flow chamber 31, and is connected to the suction channel 17 by the flow chamber 31. The second cylinder passage 41 is disposed at an upper side, extends coaxially with the first cylinder passage 40, has a smaller width than the first cylinder passage 40. An upper end of the second cylinder passage 41 has the cylinder opening 35. The tapered passage 42 is shaped with a decreasing diameter, and extends from the output end of the first cylinder passage 40 to a lower end of the second cylinder passage 41.

In FIG. 4B, a valve port sleeve 44 is included in the cylinder housing 30, positioned beside the tapered passage 42, and connected to the discharge conduit 21. The valve port sleeve 44 extends in a direction perpendicular to each of the passages 40-42 and in series with the discharge conduit 21. A flow opening 45 or seat opening for a negative pressure source is formed in an inner surface of the tapered passage 42, and at an end of the valve port sleeve 44.

A vent channel 50 is formed through the cylinder housing 30. A vent hole 47 is an outer open end of the vent channel 50 and opposed to the cap device 26. An inner hole 48 is an inner open end of the vent channel 50 and positioned inside the valve port sleeve 44. The discharge conduit 21 is open to the atmosphere through the vent channel 50.

In FIGS. 2 and 3, the plunger 25 is contained in the cylinder housing 30 slidably in a longitudinal direction, and switches the open and closed states between the suction channel 17 and the discharge conduit 21 upon operation for depressing and leaving in a non-depressed state. The plunger 25 is one piece including a plunger stem 52 and a sliding valve sleeve 53 or tapered sleeve. The plunger stem 52 extends in a longitudinal direction of the passages 40-42. The plunger stem 52 is mounted in the second cylinder passage 41 slidably in the longitudinal direction. A first stem end 52a of the plunger stem 52 protrudes from the cylinder opening 35.

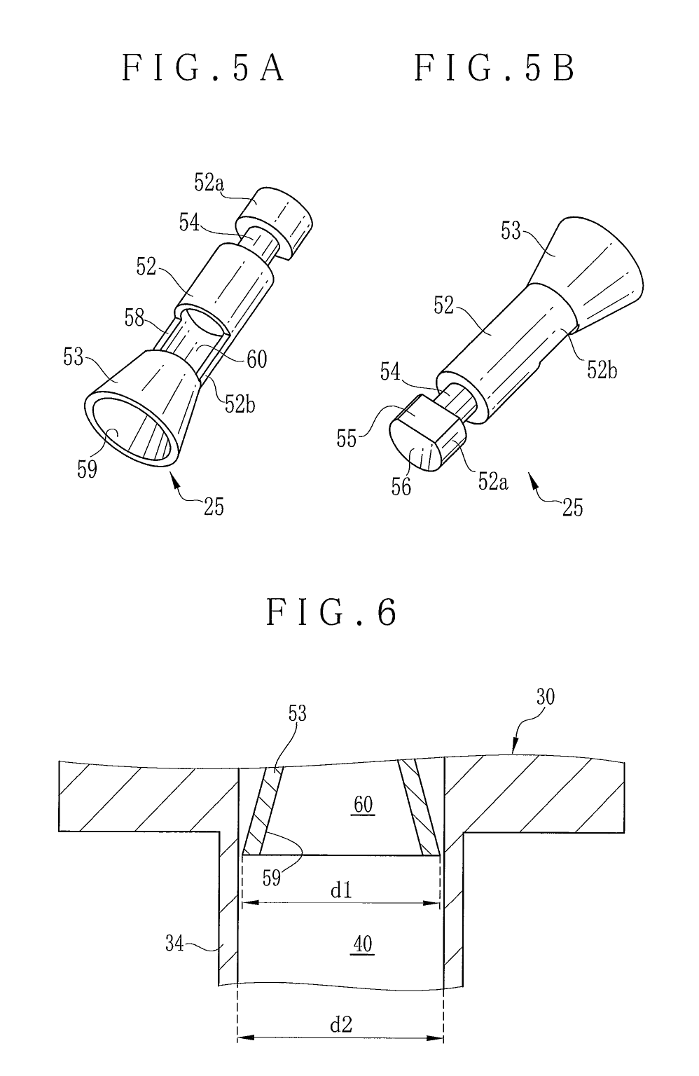

In FIGS. 5A and 5B, a cap sealing groove 54 is formed in the periphery of the first stem end 52a annularly, and used for connecting the cap device 26. A regulating surface 55 in a regulating device of FIG. 5B is formed with the first stem end 52a, is disposed higher than the cap sealing groove 54, has a flatly chamfered shape, and prevents the plunger 25 from rotating accidentally in the cap device 26. A pressure surface 56 is disposed on the first stem end 52a, and pushed manually for depression.

At a second stem end 52b of the plunger stem 52, the sliding valve sleeve 53 is disposed. An outer tapered surface of the sliding valve sleeve 53 as a valve head extends along the inner valve engaging surface of the tapered passage 42. The sliding valve sleeve 53 slides through the second cylinder passage 41 and the tapered passage 42 upon sliding of the plunger stem 52.

The plunger 25, when left without the depression, closes the flow opening 45 by contact of the sliding valve sleeve 53 with the inner valve engaging surface of the tapered passage 42. A closed state is defined by shut-off between the suction channel 17 and the discharge conduit 21. The plunger 25, when the first stem end 52a is pressed into the cylinder opening 35 with a predetermined shift by the depression, opens the flow opening 45 by setting the sliding valve sleeve 53 away from the valve engaging surface of the tapered passage 42. An open state is defined to communicate between the suction channel 17 and the discharge conduit 21. Note that the predetermined shift is defined by a position short of disabling further depression because of engagement of the cap device 26 with the upper cylinder surface 37a.

In FIGS. 5A and 5B, the plunger 25 has a valve opening 58, a flow port 59 and a flow channel 60. The valve opening 58 is formed in a side wall of the plunger stem 52. The flow port 59 is open downwards in the sliding valve sleeve 53. The flow channel 60 extends between the valve opening 58 and the flow port 59. The valve opening 58 is open in the side wall of a portion of the plunger stem 52 opposed to the flow opening 45 when the plunger 25 is in the open state. The flow channel 60, when the plunger 25 is in the open state, causes fluid in the flow port 59 to flow toward the valve opening 58, such as body fluid and mixed solid particles. A diameter of the flow channel 60 is constant in the plunger stem 52, but increases gradually in the sliding valve sleeve 53 toward the flow port 59.

In FIG. 6, a maximum outer diameter d1 of the sliding valve sleeve 53 is set smaller than an inner diameter d2 of the first cylinder passage 40. This prevents the sliding valve sleeve 53 from frictionally contacting an inner wall of the first cylinder passage 40 when the plunger 25 is slid in the cylinder housing 30. Note that the plunger stem 52 has a diameter slightly smaller than an inner diameter of the second cylinder passage 41, and is guided by the second cylinder passage 41.

In FIGS. 2 and 3, the cap device 26 is formed from elastic material such as rubber and elastomer. The cap device 26 includes a circular cap top portion 62 or cap head, and a cap skirt 63. The cap top portion 62 is coupled to the first stem end 52a. The cap skirt 63 is coupled to the distal end of the cylinder housing 30.

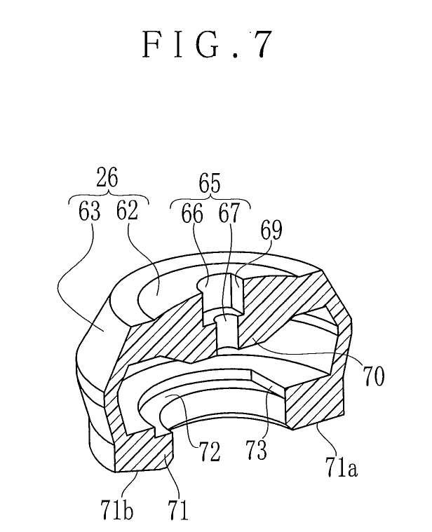

In FIG. 7, a through opening 65 is formed in the cap top portion 62 for receiving entry of the plunger 25. The through opening 65 has a receiving recess 66 and a seal hole 67 or entry hole. The receiving recess 66 is formed in an upper surface of the cap top portion 62, and receives entry of an upper part of the first stem end 52a higher than the cap sealing groove 54. The seal hole 67 receives entry of a portion of the plunger stem 52 having the cap sealing groove 54.

A regulating surface 69 in a regulating device is formed flatly by chamfering a side wall of the receiving recess 66, and firmly engaged with the regulating surface 55 of the first stem end 52a. This prevents the plunger 25 from rotating in the cap device 26. A lower hole wall 70 or first ring in the cap top portion 62 is defined by the seal hole 67, and is fitted in the cap sealing groove 54 of the first stem end 52a. Thus, the first stem end 52a is connected to the cap top portion 62.

An upper end of the cap skirt 63 is flush with the cap top portion 62. An annular ridge 71 or second ring is formed at a lower end of the cap skirt 63, and projects in an inward direction. The annular ridge 71 is fitted in the sealing groove 39. Thus, the cap device 26 is retained on the cylinder housing 30. A mating surface 71a outside the annular ridge 71 is opposed to the end surface 30a. A portion of the mating surface 71a opposed to the vent hole 47 is a beveled surface 71b, which extends with an increasing distance from the end surface 30a in a radial direction of the plunger 25.

A flange receiving recess 72 is formed in an upper wall of the annular ridge 71 around the opening, and is engaged with the bonnet flange 38. A seat positioning surface 73 in a regulating device is formed with the an inner wall of the flange receiving recess 72, has a flat shape, and is firmly engaged with the cap regulating surface 38a of the bonnet flange 38. This prevents the cap device 26 from rotating about the axis of the cylinder housing 30. Also, the plunger 25 is prevented from rotating in the cylinder housing 30 indirectly by the cap device 26. It is possible to oppose the valve opening 58 to the flow opening 45 when the plunger 25 is set in the open position.

In FIGS. 2 and 3, return force of the cap device 26 biases the plunger 25 upwards through the cylinder opening 35, to maintain the plunger 25 in the closed state. When the first stem end 52a is pressed into the cylinder opening 35, the cap top portion 62 of the cap device 26 moves toward the cylinder opening 35. In response to this, the cap skirt 63 deforms resiliently, in other words, becomes larger radially around the plunger 25 and becomes smaller (compressed) in the plunger longitudinal direction. When the suction button assembly is left without the depression, the cap device 26 returns to its original shape owing to the return force of the cap skirt 63.

At the time of the compression by the deformation, the annular ridge 71 of the cap skirt 63 is pressed toward the end surface 30a. Thus, the vent channel 50 is closed by contact of the beveled surface 71b with the vent hole 47. See FIG. 3. When the cap skirt 63 returns to its original shape, the beveled surface 71b comes away from the vent hole 47, to open the discharge conduit 21 to the atmosphere by uncovering the vent channel 50. See FIG. 2.

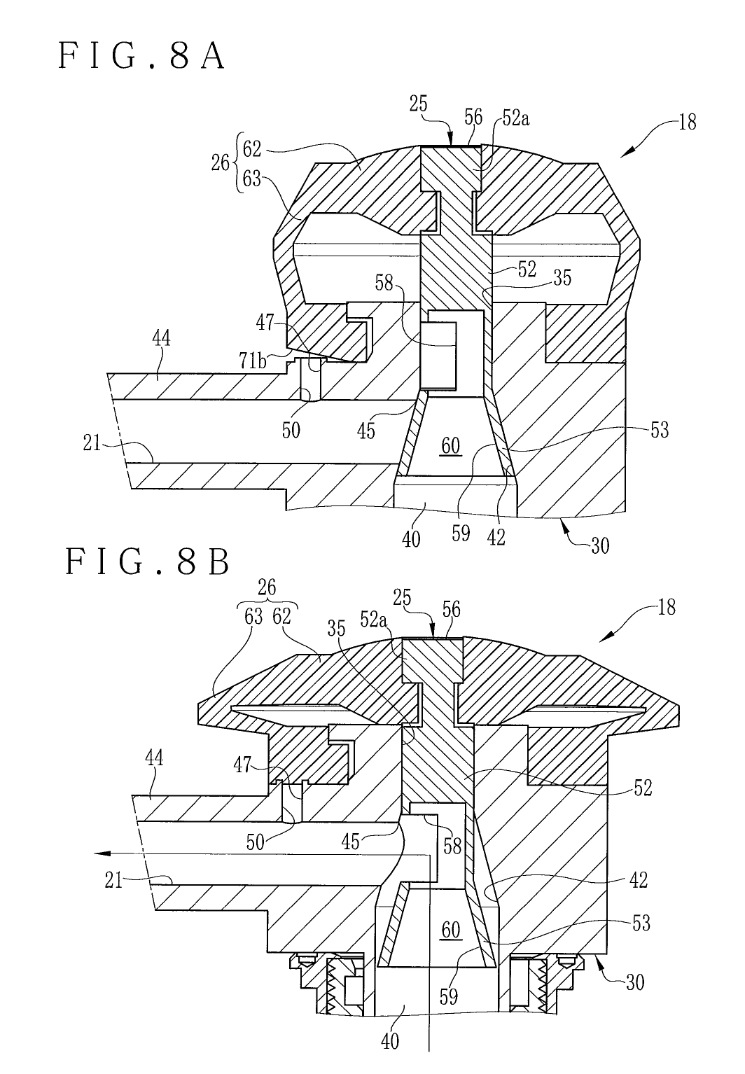

The operation of the suction button assembly 18 is described now. For diagnosis through the endoscope, the negative pressure source 20 is always used for suction. In FIG. 8A, an initial state without the suction is illustrated. The plunger 25 is set in the closed state with the cap device 26, to press the outer tapered wall (valve head) of the sliding valve sleeve 53 on the inner valve engaging surface of the tapered passage 42 or valve engaging surface. The outer wall of the sliding valve sleeve 53 tightly contacts the periphery of the flow opening 45 or seat opening, because shaped for suitable contact with the valve engaging surface of the tapered passage 42.

As the outer and inner walls are conical specifically, one of portions of the outer wall can close the flow opening 45 even when there is an error in the position or shape of the flow opening 45 or irregularity in the position of the plunger 25. The flow opening 45 can be closed tightly without a clearance space reliably. It is possible to discontinue the suction in the distal instrument opening 15 by shut-off between the suction channel 17 and the discharge conduit 21, even without attaching a seal such as an O-ring around the plunger 25 or near to the flow opening 45.

When the plunger 25 is in the closed position, the beveled surface 71b of the cap skirt 63 is away from the vent hole 47 with a clearance space. The vent channel 50 is open. Thus, the discharge conduit 21 is open to the atmosphere. It is possible to prevent occurrence of overload to the negative pressure source 20 even when the flow opening 45 is closed by the sliding valve sleeve 53.

For the suction, the pressure surface 56 is depressed as illustrated in FIG. 8B. The first stem end 52a is pushed into the cylinder opening 35 against the resiliency of the cap device 26. The cap skirt 63 of the cap device 26 is resiliently deformed to enlarge the diameter and compress vertically, so that the cap top portion 62 contacts the upper cylinder surface 37a. Thus, the plunger 25 is switched from the closed state to the open state. The sliding valve sleeve 53 shifts away from the inner valve engaging surface of the tapered passage 42 to open the flow opening 45.

When the cap skirt 63 is deformed resiliently, the vent channel 50 is closed by tight contact of the beveled surface 71b with the vent hole 47. A negative pressure or force of suction in the discharge conduit 21 increases because of its separation from the atmosphere.

When the flow opening 45 is open, the suction channel 17 becomes connected with the discharge conduit 21, to suck fluid through the distal instrument opening 15, such as body fluids, mixed solid particles and the like. The fluid flows through the suction channel 17, the valve port 32 and the flow chamber 31, is drawn into the first cylinder passage 40 of the cylinder housing 30 as indicated by the arrow, and enters the flow opening 45 upon passing the flow channel 60. The fluid in the flow opening 45 is discharged from the endoscope 10 by suction through the discharge conduit 21.

When an operator or doctor wishes to discontinue the suction, he or she leaves a thumb or finger from the pressure surface 56 for a non-depressed state. Then the cap skirt 63 recovers its initial shape by the return force, to move up the cap top portion 62 and the plunger 25. The plunger 25 is switched from the open state to the closed state. The suction channel 17 is disconnected from the discharge conduit 21 as illustrated in FIG. 8A, to discontinue the suction in the distal instrument opening 15.

Similarly, the plunger 25 is set in the open state by depression for suction, and set in the closed state by leaving in a non-depressed state for discontinuing the suction. When the plunger 25 is in the open state, relative rotation between the plunger 25 and the cylinder housing 30 is prevented by the cap device 26 indirectly. The valve opening 58 of the plunger 25 is constantly opposed to the flow opening 45. This maximizes a flow channel width between the valve opening 58 and the flow opening 45 for flow of fluid. The suction through the suction button assembly 18 is the highest.

As the cap device 26 in the suction button assembly 18 indirectly prevents the plunger 25 from rotating in the cylinder housing 30, it is unnecessary to dispose an anti-rotation structure between the plunger 25 and an inner surface of the cylinder housing 30. This is effective in preventing occurrence of resistance in frictional contact in an anti-rotation structure between the plunger 25 and the cylinder housing 30 while the plunger 25 slides in the cylinder housing 30. Consequently, the plunger 25 can return to the closed state reliably without failure, because this resistance in frictional contact can be suppressed.

The sliding valve sleeve 53 has a maximum outer diameter d1 smaller than an inner diameter d2 of the first cylinder passage 40. The sliding valve sleeve 53 is pressed against the inner valve engaging surface of the tapered passage 42 in the closed state, but does not contact the valve engaging surface and the second cylinder passage 41 in a state other than the closed state. As a result, the sliding valve sleeve 53 slides without frictional contact with the tapered passage 42 and the second cylinder passage 41. It is possible to suppress occurrence of resistance of contact between the plunger 25 and the inner wall of the cylinder housing 30, to prevent occurrence of failure reliably.

Another preferred suction button assembly 80 or shut-off valve assembly of the invention is described now. Although the suction button assembly 18 of the above embodiment has the flow opening 45 in the tapered passage 42 in the cylinder housing 30, the suction button assembly 80 has a flow opening in a higher portion near to the cylinder opening 35 than a conically tapered passage in a cylinder housing 30.

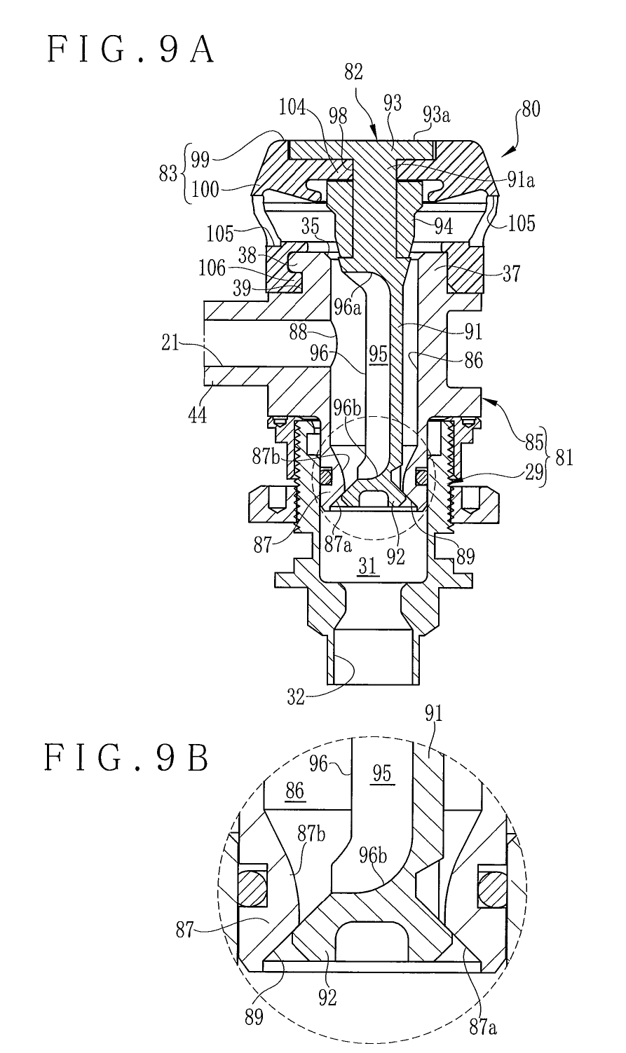

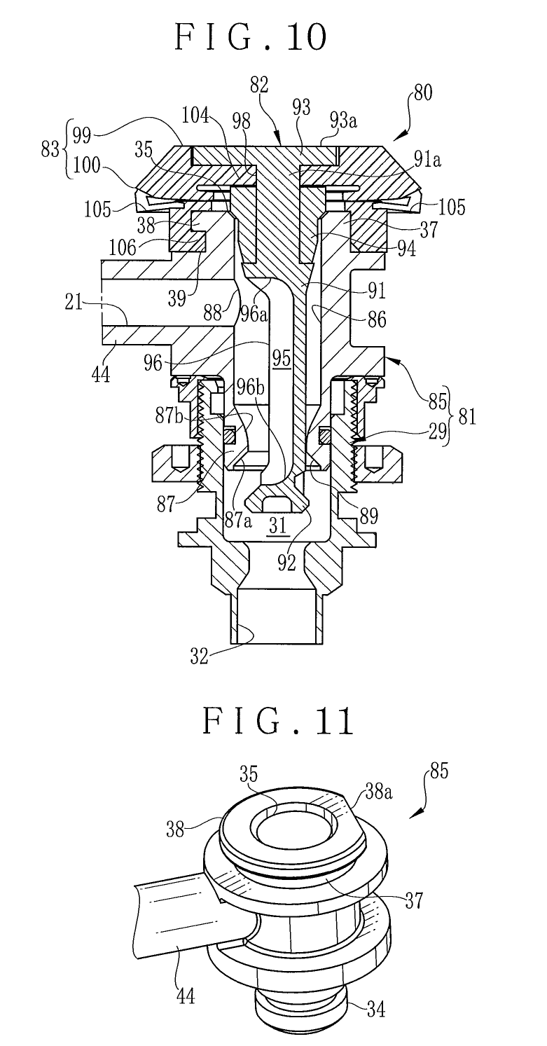

In FIGS. 9A, 9B and 10, the suction button assembly 80 includes a housing device 81 or cylinder device, a plunger 82, and a cap device 83. The plunger 82 is contained in the housing device 81. The cap device 83 connects the plunger 82 to the housing device 81. Elements similar to the above embodiment are designated by identical reference numerals.

The housing device 81 includes the support sleeve 29 and a cylinder housing 85 or valve guide fixedly connected with the support sleeve 29. For the cylinder housing 85, the cylinder housing 30 is repeated with differences in that there are a straight cylinder passage 86 (cylinder bore) and a flow sleeve 87. The cylinder passage 86 extends coaxially with the flow chamber 31, and has the cylinder opening 35 at its upper end. The flow sleeve 87 extends from an input end of the cylinder passage 86. Note that the vent channel 50 described above does not exist in the cylinder housing 85.

A flow opening 88 for a negative pressure source is open at the cylinder passage 86, and communicates with the discharge conduit 21. A valve seat port 89 is open at a lower cylinder end of the flow sleeve 87, and has a first conically tapered passage 87a or valve engaging surface, and a second conically tapered passage 87b. The first tapered passage 87a has a diameter decreasing toward the cylinder opening 35 gradually. The second tapered passage 87b has a diameter increasing toward the cylinder opening 35 gradually, and connects an output end of the first tapered passage 87a to the input end of the cylinder passage 86.

In FIG. 11, the bonnet flange 38 is disposed at an end of the cylinder sleeve 37 of the cylinder housing 85 in a manner similar to the cylinder housing 30 of the first embodiment. The cap regulating surface 38a is formed with the bonnet flange 38.

In FIGS. 9A, 9B and 10, the plunger 82 is one molded piece including a plunger stem 91, a sliding valve head 92, and a pressure head 93. The plunger stem 91 is contained in the cylinder passage 86, is cylindrical and axially long, and has a smaller diameter than the cylinder passage 86. The sliding valve head 92 is disposed at a lower stem end (second) of the plunger stem 91. There is a first stem end 91a of the plunger stem 91 protruding from the cylinder opening 35. The pressure head 93 is disposed at the first stem end 91a. The sliding valve head 92 is shaped to extend along an inner wall of the first tapered passage 87a.

A spacer ring 94 is secured to the first stem end 91a for entry in the cylinder opening 35 when pressed therein by manual depression. The spacer ring 94 has a conical surface with a diameter increasing upwards away from the cylinder opening 35. A pressure surface 93a is defined at a top of the pressure head 93 for receiving force of depression.

In the plunger 82 constructed above, the sliding valve head 92 closes the valve seat port 89 upon leaving the suction button assembly without the depression, because the sliding valve head 92 contacts an inner wall of the first tapered passage 87a. A closed state is defined for shut-off between the suction channel 17 and the discharge conduit 21. See FIG. 9B. When the first stem end 52a of the plunger 82 is pressed into the cylinder opening 35 with a predetermined shift by the depression, the sliding valve head 92 comes away from the first tapered passage 87a to open the valve seat port 89. An open state is defined to communicate between the suction channel 17 and the discharge conduit 21. See FIG. 10. Note that the predetermined shift is defined by a position short of disabling further depression because of engagement of the spacer ring 94 with the cylinder opening 35.

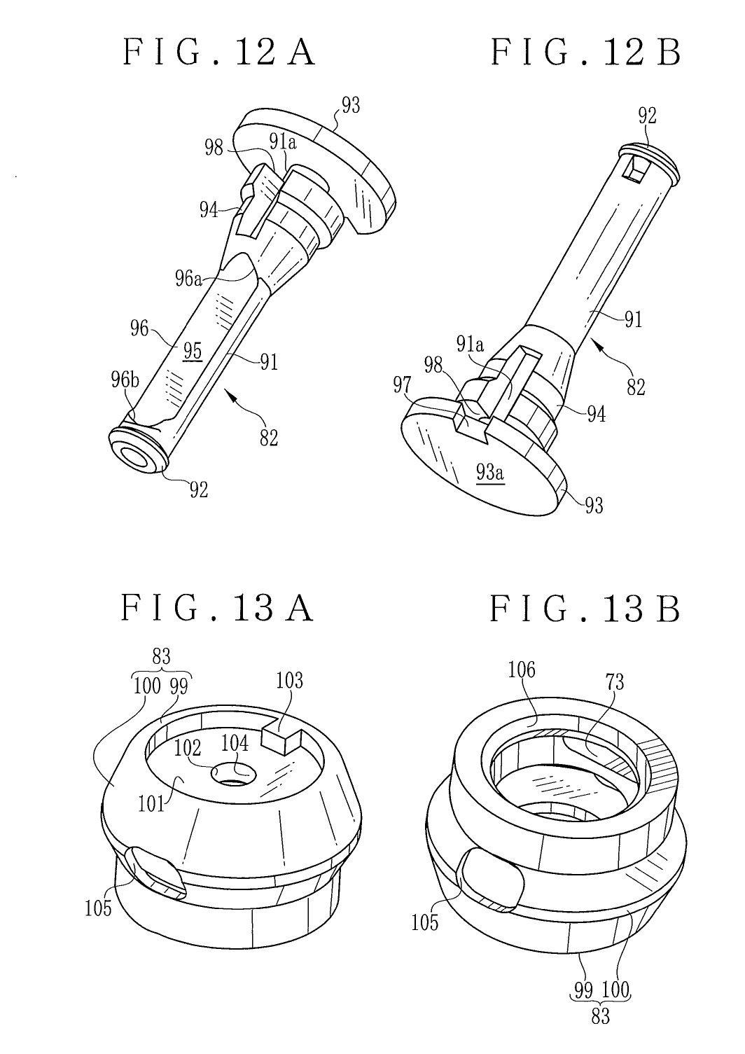

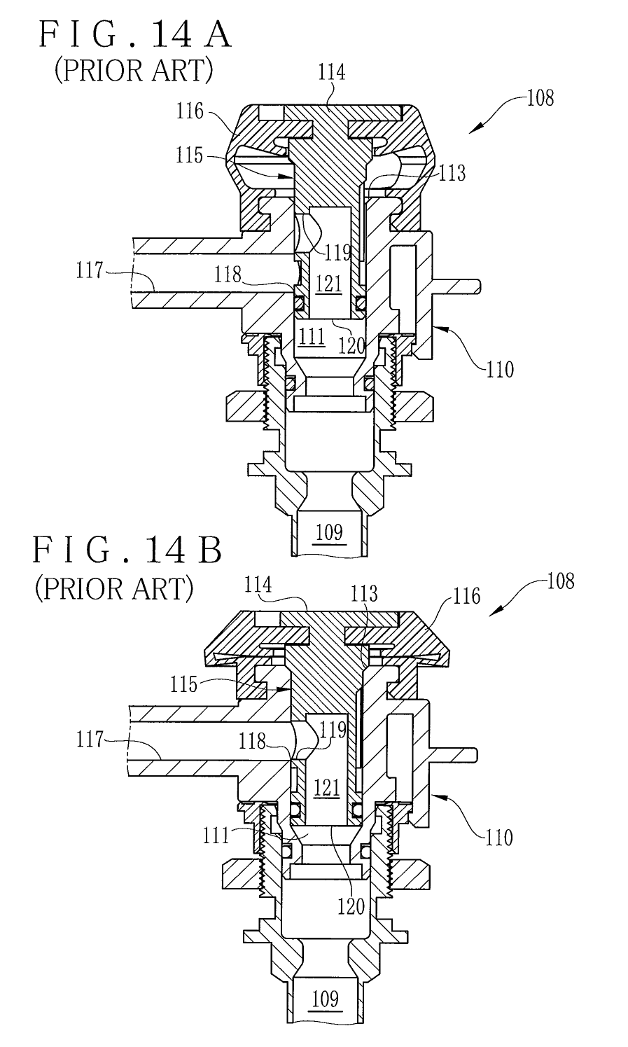

In FIGS. 12A and 12B, a longitudinal cutout 95 is formed in the plunger stem 91 and extends longitudinally. A flow channel 96 of a linear shape is defined by the longitudinal cutout 95, and positioned with the plunger stem 91. When the plunger 82 is set in the open state, an output channel surface 96a of the flow channel 96 is opposed to the flow opening 88. An input channel surface 96b of the flow channel 96 is positioned on a side nearer to the suction channel 17 than the flow sleeve 87. In consideration of those, a length of the longitudinal cutout 95 is predetermined.

The input channel surface 96b is an entrance for drawing fluid from the flow chamber 31 to the longitudinal cutout 95 in the open state. The output channel surface 96a is an exit for drawing out fluid from the longitudinal cutout 95 in the open state.

A regulating recess 97 in a regulating device is formed by chamfering a peripheral wall of the pressure head 93, and prevents the cap device 83 from rotating. A sealing groove 98 of FIGS. 9 and 10 is defined annularly between the pressure head 93 and the spacer ring 94, and used for connecting the cap device 83.

In FIGS. 13A and 13B, the cap device 83 is shaped similarly to the cap device 26, and includes a circular cap top portion 99 or cap head, and a cap skirt 100. The cap top portion 99 is connected to the pressure head 93. The cap skirt 100 is disposed around the cap top portion 99 and the first stem end 91a, and is connected to a distal end of the cylinder housing 85.

A receiving recess 101 and a seal hole 102 or entry hole are formed in the cap top portion 99. The receiving recess 101 receives entry of the pressure head 93. The seal hole 102 receives entry of the first stem end 91a. A regulating projection 103 in a regulating device projects from an inner surface of the receiving recess 101, and is engaged with the regulating recess 97 of the pressure head 93. A hole wall 104 or first ring is defined by the seal hole 102, and is fitted in the sealing groove 98 of the first stem end 91a.

A vent opening 105 is formed in the cap skirt 100. An annular ridge 106 or second ring projects from a lower end of the cap skirt 100 and is fitted in the sealing groove 39. The flange receiving recess 72 of FIG. 7 is formed in an upper surface of the annular ridge 106, which has been described with the first embodiment. The seat positioning surface 73 is formed on an inner wall of the receiving recess 101. Those prevent the cap device 83 from rotating about the axis of the cylinder housing 85. Also, the cap device 83 indirectly prevents the plunger 82 from rotating in the cylinder housing 85. It is possible with their rotational regulation to oppose the output channel surface 96a to the flow opening 88 when the plunger 82 is set in the open state.

The operation of the suction button assembly 80 is described now. In FIGS. 9A and 9B, an initial state without the suction is illustrated. The plunger 82 is set in the closed state with the cap device 83, to press an outer wall of the sliding valve head 92 on an inner wall of the first tapered passage 87a or valve engaging surface. The valve seat port 89 is closed. The outer wall of the sliding valve head 92 is tapered for suitable contact with the tapered inner wall of the first tapered passage 87a. In a manner similar to the first embodiment, the valve seat port 89 can be closed reliably without attaching a seal structure to the outside of the plunger 82 or the inside of the first tapered passage 87a, such as an O-ring. It is possible to discontinue the suction in the distal instrument opening 15 by shut-off between the suction channel 17 and the discharge conduit 21.

When the plunger 82 is in the closed state, an annular clearance space is created between an outer surface of the plunger stem 91 and the cylinder opening 35, for the flow opening 88 to communicate with the vent opening 105 of the cap device 83. This is effective in preventing occurrence of overload to the negative pressure source 20 because the discharge conduit 21 is open to the atmosphere.

For the suction, the pressure surface 93a is depressed as illustrated in FIG. 10. The first stem end 91a is pushed into the cylinder opening 35. The cap skirt 100 of the cap device 83 is resiliently deformed to enter the spacer ring 94 in the cylinder opening 35. Thus, the plunger 82 is switched from the closed state to the open state. The sliding valve head 92 shifts away from the inner wall of the first tapered passage 87a to open the valve seat port 89. As the cylinder opening 35 is closed by the spacer ring 94, a negative pressure or force of suction in the discharge conduit 21 increases because of its separation from the atmosphere.

When the valve seat port 89 is open, the suction channel 17 communicates with the discharge conduit 21. Fluid drawn through the distal instrument opening 15 flows through the suction channel 17 and the flow chamber 31 and into the longitudinal cutout 95 and the cylinder passage 86 after passing the input channel surface 96b. Then the fluid enters the flow opening 88 upon passing the cylinder passage 86 and the longitudinal cutout 95. The fluid in the flow opening 88 passes the discharge conduit 21 and is drawn by external suction through the discharge conduit 21 to the outside of the endoscope 10.

When set in the open state, the plunger 82 is prevented by the cap device 83 from rotating about the axis of the cylinder housing 85. The input channel surface 96b in the plunger 82 can be kept opposed to the flow opening 88. This maximizes a flow channel width between the valve seat port 89 and the flow opening 88 for flow of fluid. The suction through the suction button assembly 80 is the highest similarly to the first embodiment.

To discontinue the suction, the pressure surface 93a is released from the depression. The return force of the cap skirt 100 slides the plunger 82 to the closed state, to discontinue the suction through the distal instrument opening 15.

As the plunger 82 is indirectly prevented from rotating in the cylinder housing 85, it is unnecessary to add an anti-rotation structure between the plunger 82 and the cylinder housing 85. This is effective in preventing occurrence of resistance in frictional contact of the anti-rotation structure for the plunger 82 and the cylinder housing 85 while the plunger 82 slides in the cylinder housing 85. Consequently, the plunger 82 can return to the closed state reliably without failure.

In the above embodiments, the regulating surface 55 and the regulating recess 97 are used for rotational regulation of the plungers 25 and 82. The regulating surface 69 and the regulating projection 103 are used for rotational regulation of the cap devices 26 and 83. However, any of well-known preventing structures for rotational regulation at plungers or cap devices can be used without limitation.

In the above embodiments, the cap regulating surface 38a is associated with the cylinder housings 30 and 85 as a regulating structure. The seat positioning surface 73 is associated with the cap devices 26 and 83 as a regulating structure. However, types and arrangements of regulating structures of the invention are not limited for the purpose of rotational regulation between a cylinder housing and a cap device.

In the above embodiments, the tapered passage 42 and the first tapered passage 87a (or valve engaging surface) are formed in the cylinder housings 30 and 85 as tapered passages of the invention. However, positions and shapes of passages of the invention are not limited. It is possible to modify positions and shapes of the valve elements of the plungers 25 and 82 according to a change in the tapered passages.

In the above embodiments, the plungers 25 and 82 are kept in the closed state by the return force of the skirts 63 and 100 of the cap devices 26 and 83. However, other structures can be used for keeping the plungers 25 and 82 in the closed state, such as a compression coil spring disposed between the cap top portion 62, 99 and the cylinder housing 30, 85 for biasing the plunger 25, 82.

In the above embodiments, the support sleeve 29 is separate from the cylinder housings 30 and 85 to constitute the housing device 24 and 81. However, the support sleeve 29 may be formed with the cylinder housing 30 or 85 by way of a single housing device.

In the above embodiments, the endoscope 10 with the suction button assembly 18 or 80 is a bronchoscope. However, the endoscope 10 of the invention may be an endoscope of other type, such as a colonoscope.

Also, the suction button assembly of the invention can be so constructed with an input end of a flow path disposed on a lateral side and an output end of the flow path disposed on a lower side, that the suction channel 17 and the discharge conduit 21 can be connected in a reverse manner.

In the above cap device, the through opening 65 is formed in the cap top portion 62 for receiving entry of the plunger 25. However, the cap top portion 62 may not have a through opening. Only a center recess can be formed in a lower wall of the cap top portion 62 for receiving entry of the upper end of the plunger 25.

It is possible with the feature of in the invention to combine various known structures of relevant fields of the catheter combined with a valve, and the like.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A suction button assembly for an endoscope including an operation unit, a section of an elongated tube for entry in a body cavity, and a suction channel and a discharge conduit disposed with said operation unit, wherein a distal opening is formed in a distal end of said elongated tube, said suction channel is disposed to extend to said distal opening, said discharge conduit is connected to a negative pressure source, said suction button assembly comprising:
a cylinder housing, disposed on said operation unit, and including a cylinder passage, a valve engaging surface and a flow opening, said cylinder passage being formed through said cylinder housing, said valve engaging surface being disposed in said cylinder passage, said flow opening formed in a wall of said cylinder passage, said cylinder passage having first and second cylinder openings, said first cylinder opening being connected to said suction channel, said valve engaging surface being tapered with an inner diameter decreasing from said first cylinder opening toward said second cylinder opening, said discharge conduit extending from said flow opening;
a plunger, contained in said cylinder passage in a slidable manner, and including a stem end, a valve head and a flow channel, said stem end protruding from said second cylinder opening, said valve head being movable to and from said valve engaging surface for contact, said flow channel being opposed to said flow opening, wherein said plunger is in a closed position when said stem end is free from being depressed, and is in an open position when said stem end is depressed, and said valve head, when said plunger is in said closed position, contacts said valve engaging surface for interruption between said suction channel and said discharge conduit, and when said plunger is in said open position, leaves from said valve engaging surface for communication between said suction channel and said discharge conduit in cooperation with said flow channel aligned with said flow opening;
a cap device, secured to said stem end and said cylinder housing, for covering said stem end;
a first regulating device for preventing said plunger from rotating in said cap device; and
a second regulating device for preventing said cap device from rotating about an axis of said cylinder housing, whereby keeping said flow channel aligned with said flow opening.

2. A suction button assembly as defined in claim 1, wherein said first regulating device includes:
a first regulating portion formed with said plunger;
a second regulating portion, formed with said cap device, for engagement with said first regulating portion.

3. A suction button assembly as defined in claim 2, wherein said first regulating portion is a recess portion formed in said plunger, and said second regulating portion is a projection disposed to project from an inner surface of said cap device toward said recess portion.

4. A suction button assembly as defined in claim 2, wherein said second regulating device includes:
a third regulating portion formed with said cylinder housing;
a fourth regulating portion, formed with said cap device, for engagement with said third regulating portion.

5. A suction button assembly as defined in claim 4, wherein said third regulating portion is a recess portion formed in said cylinder housing, and said fourth regulating portion is a projection disposed to project from an inner surface of said cap device toward said recess portion.

6. A suction button assembly as defined in claim 4, wherein said flow opening is formed in said valve engaging surface, and when said plunger is in said closed position, is closed by said valve head, and when said plunger is in said open position, is open upon separation of said valve head.

7. A suction button assembly as defined in claim 6, wherein said flow channel is formed through said plunger, and includes:
a flow port, formed at an end of said plunger, and open in said cylinder passage;
a valve opening, formed in a wall of said plunger, and opposed to said flow opening when said plunger is in said open position.

8. A suction button assembly as defined in claim 7, wherein said valve head is disposed at an end of said plunger opposite to said stem end;
said flow channel has an inner diameter increasing toward said flow port in said valve head.

9. A suction button assembly as defined in claim 8, wherein said cylinder passage further includes:
a first cylinder passage, disposed between said valve engaging surface and said first cylinder opening, and connected to said suction channel;
a second cylinder passage, disposed between said valve engaging surface and said second cylinder opening, having a smaller inner diameter than said first cylinder passage, for containing said stem end in a slidable manner.

10. A suction button assembly as defined in claim 9, wherein a maximum outer diameter of said valve head is smaller than an inner diameter of said first cylinder passage.

11. A suction button assembly as defined in claim 4, wherein said flow opening is disposed between said valve engaging surface and said second cylinder opening;
said flow channel is formed between said valve head and said stem end, and kept opposed to said flow opening.

12. A suction button assembly as defined in claim 11, wherein said valve head is disposed at an end of said plunger opposed to said stem end;
said flow channel is a longitudinal cutout formed linearly to extend in a plunger longitudinal direction;
said cylinder passage is cylindrical with one inner diameter.

13. A suction button assembly as defined in claim 4, further comprising a vent channel, formed in said cylinder housing, disposed to extend to said discharge conduit, opened for venting of said discharge conduit when said plunger is in said closed position, and closed by said cap device when said plunger is in said open position.

14. A suction button assembly as defined in claim 4, wherein a vent opening is formed in said cap device, and a gap is formed between said second cylinder opening and said plunger when said plunger is in said open position, for venting through said vent opening in said cap device, and said gap is closed by said plunger when said plunger is in said closed position.

15. A suction button assembly as defined in claim 4, wherein said cap device is formed from resilient material and deformable resiliently upon slide of said plunger.

16. A suction button assembly as defined in claim 15, wherein said cap device includes:
a cap top portion of a disk shape; and
a cap skirt, formed to project in a downward direction from an edge of said cap top portion, having a lower end secured to said cylinder housing, for compressing in said downward direction when depressing force is exerted on said plunger to said open position, and for extending with return force thereof when said plunger is released from said depressing force, to return said plunger to said closed position.

17. A suction button assembly as defined in claim 15, further comprising:
an upper cylinder surface, disposed on said cylinder housing, and opposed to said cap device;
a lower wall, disposed under said cap device, received by said upper cylinder surface upon sliding of said plunger stem in said downward direction, for preventing sliding thereof beyond a predetermined shift.

18. A suction button assembly as defined in claim 4, wherein said negative pressure source is a suction pump.

19. A suction button assembly of a valve structure for an endoscope, comprising:
a cylinder housing;
a cylinder passage formed through said cylinder housing;
a plunger, contained in said cylinder passage, and slidable between open and closed positions;
a suction channel formed with said cylinder housing to extend in an upstream direction from said cylinder passage;
a discharge conduit formed with said cylinder housing to extend in a downstream direction from said cylinder passage;
a flow opening formed in a wall of said cylinder passage, said discharge conduit extending from said flow opening;
a flow channel, formed in said plunger, for communicating said suction channel with said discharge conduit through said flow opening when said plunger is in said open position;
a cap device, secured to an upper end of said plunger, for covering said plunger over said cylinder housing;
a first regulating device for preventing said plunger from rotating in said cap device;
a second regulating device for preventing said cap device from rotating about an axis of said cylinder housing, to keep said flow channel aligned with said flow opening.
